(19) United States
(12) Patent Application Publication
(10) Pub. No.: US 2023/0250125 A1
(43) Pub. Date: Aug. 10, 2023

(54) NUCLEIC ACID POLYMERS

(71) Applicant: JANSSEN BIOPHARMA, INC., South San Francisco, CA (US)

(72) Inventors: Sergei GRYAZNOV, Beerse (BE); Lakshmipathi PANDARINATHAN, Beerse (BE)

(21) Appl. No.: 17/995,073

(22) PCT Filed: Mar. 31, 2021
(86) PCT No.: PCT/IB2021/052704

§ 371 Date: Sep. 29, 2022

Related U.S. Application Data
(60) Provisional application No. 63/003,814, filed on Apr. 1, 2020.

(51) Int. Cl. C07H 21/00; A61P 31/14

(52) U.S. Cl. CPC C07H 21/00; A61P 31/14

(57) ABSTRACT

Modified nucleic acid polymers comprising modifications at the 2′ and/or 3′ positions(s) along with methods of making and use, e.g., inhibiting viral activity are disclosed.

### INCORPORATION BY REFERENCE TO ANY PRIORITY APPLICATIONS

Any and all applications for which a foreign or domestic priority claim is identified, for example, in the Application Data Sheet or Request as filed with the present application, are hereby incorporated by reference under 37 CFR 1.57, and Rules 4.18 and 20.6, including U.S. Provisional Application No. 63/003,814, filed Apr. 1, 2020.

### BACKGROUND

Random sequence nucleic acid polymers (NAPs) that have antiviral activity have been considered for use as antiviral agents, for example, in the treatment or prevention of various diseases and conditions such as viral infections or diseases. These oligonucleotides can demonstrate non-sequence complementary antiviral activity, which is believed to be driven by interactions with large amphipathic protein domains important for viral replication. NAP activity is believed to rely on the length of the oligonucleotide and the presence of phosphorothioation. Roehl, Ingo, et al. “Nucleic acid polymers with accelerated plasma and tissue clearance for chronic hepatitis B therapy.” Molecular Therapy-Nucleic Acids 8 (2017): 1-12.

Recent studies have shown that a specific NAP (e.g., REP 2139 and REP 2055) can block the release of HBsAg and reduce intracellular HBsAg. However, this compound remains in clinical trials, and there is a need to provide new compounds with improved therapeutic profile and/or reduced toxicities. The present disclosure addresses these unmet needs by providing compounds that demonstrate superior potency.

### SUMMARY

The present disclosure relates to compounds and compositions containing NAPs and methods of use and manufacture of these compounds and compositions.

Certain embodiments include a nucleic acid polymer comprising 8 to 50 nucleoside subunits linked by intersubunit linkages, wherein the nucleic acid polymer comprises (A) one or more 3′-5′ thiophosphoramidate intersubunit linkage and the remaining intersubunit linkages are 3′-5′ thiophosphate intersubunit linkages and/or (B) at least 40% of the nucleoside subunits contain a 2′-MOE substituent. In some embodiments, the nucleic acid polymer comprises five or more 3′-5′ thiophosphoramidate intersubunit linkages and the remaining intersubunit linkages are 3′-5′ thiophosphate intersubunit linkages. In some embodiments, the nucleic acid polymer, wherein half the intersubunit linkages are 3′-5′ thiophosphoramidate intersubunit linkages and the remaining intersubunit linkages are 3′-5′ thiophosphate intersubunit linkages. In some embodiments, the nucleoside subunits each independently contain a nucleobase selected from adenine, guanine, cytosine, 5-methylcytosine, and uracil. In some embodiments, the nucleobase is selected from adenine, cytosine, and 5-methylcytosine. In some embodiments, the nucleobase is selected from adenine, cytosine, and 5-methylcytosine. In some embodiments, the nucleoside subunits are substituted at the 2′ position with OMe or MOE.

In some embodiments, the nucleic acid polymer is represented by the following formula (I):

(N¹-L¹-N²-L²-N³-L³-N⁴-L⁴)ₓ (I)

wherein N¹ and N³ represent a nucleoside with a nucleobase selected from adenine, guanine, cytosine, 5-methylcytosine, and uracil; N² and N⁴ represent a nucleoside with a nucleobase selected from adenine, guanine, cytosine, 5-methylcytosine, and uracil; L¹, L², L³ and L⁴ each independently are a ps or nps linkage, and at least one is a nps linkage; and x is an integer from 2 to 16. In some embodiments, N¹, N², N³ and N⁴ are each independently substituted at the 2′ position with OMe or MOE. In some embodiments, the nucleobase of N¹ and N³ is adenine and the nucleobase of N² and N⁴ is cytosine or 5-methylcytosine. In some embodiments, one of L¹ and L² is nps. In some embodiments, two of L¹, L², L³ and L⁴ is nps. In some embodiments, x is an integer from 2 to 10.

In some embodiments, the nucleic acid polymer is represented by the following formula (II):

(N⁵-L⁵-N⁶-L⁶)_{y} (II)

wherein N⁵ and N⁶ represent a nucleoside with a nucleobase selected from adenine, guanine, cytosine, 5-methylcytosine, and uracil; L⁵ and L⁶ each independently are a ps or nps linkage, and at least one is a nps linkage; and x is an integer from 4 to 22. In some embodiments, N⁵ and N⁶ are each independently substituted at the 2′ position with OMe or MOE. In some embodiments, the nucleobase of N⁵ is adenine and the nucleobase of N⁶ is cytosine or 5-methylcytosine. In some embodiments, L⁵ and L⁶ are each nps. In some embodiments, x is an integer from 4 to 20. In some embodiments, the nucleic acid polymer is conjugated to a ligand targeting group and/or chelating group. In some embodiments, the nucleic acid polymer is selected from: (mXnpsmXpsmXpsmXps)10; (mXpsmXnpsmXpsmXps)10; (mXpsmXpsmXnpsmXps)10; (mXpsmXpsmXpsmXnps)10; (XnpsXnps)5-15; and (moeXpsmXps)20, wherein X is independently in each instance a nucleoside having natural or modified nucleobase.

Other embodiments include a pharmaceutical composition comprising a nucleic acid polymer of any one of the disclosed embodiments and a pharmaceutically acceptable excipient.

Other embodiments include a method for treating a subject having a viral infection comprising administering to the subject in need thereof a therapeutically effective amount of the nucleic acid polymer of any one of the disclosed embodiments. In some embodiments, the viral infection is from Hepatitis B virus and/or Hepatitis D virus. In some embodiments, the subject is a mammal. In some embodiments, the mammal is a human. In some embodiments, the nucleic acid polymer is administered as part of a co-treatment with at least one additional therapeutic agent. In some embodiments, the at least one additional therapeutic agent comprises at least one selected from an antisense oligonucleotide and RNAi.

Certain embodiments include a method inhibiting viral activity in a subject comprising administering to the subject in need thereof a therapeutically effective amount of the nucleic acid polymer of any one of the disclosed embodiments. In some embodiments, the viral activity is from Hepatitis B virus and/or Hepatitis D virus. In some embodiments, the subject is a mammal. In some embodiments, the mammal is a human.

Certain embodiments include the nucleic acid polymer of any one the disclosed embodiments for use in the treatment of a viral infection. Certain embodiments include use of a nucleic acid polymer of any one of the disclosed embodiments for the manufacture of a medicament for treatment of a viral infection.

### DETAILED DESCRIPTION

The present disclosure is directed to compounds and compositions containing NAPs and methods of use and manufacture of these compounds and compositions.

### Naps

NAPs of the present disclosure include modified nucleotides with particular 2′ and 3′ modifications. For example, the NAPs may include nucleoside subunits linked by intersubunit linkages. In some embodiments, the nucleoside is modified at the 2′ position of the sugar ring, e.g., with —O(CR′₂)₀₋₂ O(CR′₂)₀₋₁CR′₃, where R′ is independently in each instance H or F. In some embodiments, the nucleoside is modified at the 3′ position of the sugar ring, e.g., with NH.

In certain embodiments, the NAPs of the present disclosure comprise 8 to 50 nucleotides (nucleoside subunits linked by intersubunit linkages), and have one or more 3′-5′ thiophosphoramidate intersubunit linkage and the remaining intersubunit linkages are 3′-5′ thiophosphate intersubunit linkages. In some embodiments, the NAP can have, e.g., 1, 2, 3, 4, 5, 6, 7, 8, 9, 10, 11, 12, 13, 14, 15, 16, 17, 18, 19, 20, or more thiophosphoramidate intersubunit linkages. In some embodiments, the NAP can have, e.g., 25%, 50%, 75% or 100% thiophosphoramidate intersubunit linkages. In some embodiments, the NAP can have a pattern of thiophosphoramidate intersubunit linkages, e.g., every second, third, fourth, fifth, sixth, seventh or eighth intersubunit linkage is a thiophosphoramidate intersubunit linkage.

In certain embodiments, the NAPs of the present disclosure comprise 8 to 50 nucleotides (nucleoside subunits linked by intersubunit linkages), where the nucleoside is modified at the 2′ position of the sugar ring, e.g., with —O(CR′₂)₀₋₂ O(CR′₂)₀₋₁CR′₃, where R′ is independently in each instance H or F. In some embodiments, the NAP can have, e.g., 1, 2, 3, 4, 5, 6, 7, 8, 9, 10, 11, 12, 13, 14, 15, 16, 17, 18, 19, 20, 25, 30, 35, 40 or more 2′-modified nucleosides. In some embodiments, the NAP can have, e.g., 25%, 30%, 35%, 40%, 45%, 50%, 75% or 100% 2′-modified nucleosides. In some embodiments, the 2′-modified nucleosides is substituted with 2′-OMe or 2′-MOE. In some embodiments, the NAP can have a pattern of alternating 2′-OMe or 2′-MOE substitutions. In other embodiments, every nucleoside is 2′ modified, e.g., every nucleoside has 2′-OMe or 2′-MOE substitution. In some embodiments, all but 1, 2, 3, 4 or 5 nucleosides are 2′ modified.

In certain embodiments, the nucleoside subunits of the NAP comprise a natural or modified nucleobase. For instance, in some embodiments, the nucleoside subunits comprise a nucleobase selected from adenine, guanine, cytosine, 5-methylcytosine, and uracil. In some embodiments, the nucleoside subunits comprise a nucleobase selected from cytosine and 5-methylcytosine. In some embodiments, the NAP can have a pattern of alternating nucleobases selected from adenine, guanine, cytosine, 5-methylcytosine, and uracil, e.g., alternating adenine and 5-methylcytosine.

In certain embodiments, the NAP is represented by the following formula (I):

(N¹-L¹-N²-L²-N³-L³-N⁴-L⁴)ₓ(I)

wherein

N¹ and N³ represent a nucleoside with a natural or modified nucleobase (e.g., a nucleobase selected from adenine, guanine, cytosine, 5-methylcytosine, and uracil);

N² and N⁴ represent a nucleoside with a natural or modified nucleobase (e.g., a nucleobase selected from adenine, guanine, cytosine, 5-methylcytosine, and uracil);

L¹, L², L³ and L⁴ each independently are a ps or nps linkage, and at least one is a nps linkage; and

x is an integer from 2 to 16.

In some embodiments, N¹, N², N³ and N⁴ are each independently substituted at the 2′ position with OMe or MOE. In some embodiments, the nucleobase of N¹ and N³ is adenine and the nucleobase of N² and N⁴ is cytosine or 5-methylcytosine. In some embodiments, one of L¹ and L² is nps (e.g., L¹ is nps or L² is nps). In other embodiments, two or three of L¹, L², L³ and L⁴ is nps. In some embodiments, x is an integer from 2 to 16, e.g., 2, 3, 4, 5, 6, 7, 8, 9, 10, 11, 12, 13, 14, 15 or 16.

Other embodiments, include a NAP represented by the following formula (II):

(N⁵-L⁵-N⁶-L⁶)_{y} (II)

wherein

N⁵ and N⁶ represent a nucleoside with a natural or modified nucleobase (e.g., a nucleobase selected from adenine, guanine, cytosine, 5-methylcytosine, and uracil);

L⁵ and L⁶ each independently are a ps or nps linkage, and at least one is a nps linkage; and

x is an integer from 4 to 22.

In some embodiments, N⁵ and N⁶ are each independently substituted at the 2′ position with OMe or MOE. In some embodiments, the nucleobase of N⁵ is adenine and the nucleobase of N⁶ is cytosine or 5-methylcytosine. In some embodiments, L⁵ and L⁶ are each nps or L⁵ is nps or L⁶ is nps. In some embodiments, x is an integer from 2 to 16, e.g., 4, 5, 6, 7, 8, 9, 10, 11, 12, 13, 14, 15, 16, 17, 18, 19, 20, 21 or 22.

In some embodiments, the NAP is selected from:

(mXnpsmXpsmXpsmXps)2-16, e.g., 2, 3, 4, 5, 6, 7, 8, 9, 10, 11, 12, 13, 14, 15 or 16;

(mXpsmXnpsmXpsmXps) 2-16, e.g., 2, 3, 4, 5, 6, 7, 8, 9, 10, 11, 12, 13, 14, 15 or 16;

(mXpsmXpsmXnpsmXps) 2-16, e.g., 2, 3, 4, 5, 6, 7, 8, 9, 10, 11, 12, 13, 14, 15 or 16;

(mXpsmXpsmXpsmXnps) 2-16, e.g., 2, 3, 4, 5, 6, 7, 8, 9, 10, 11, 12, 13, 14, 15 or 16;

(XnpsXnps)5-15; and

(moeXpsmXps)20,

wherein X is independently in each instance a nucleoside having natural or modified nucleobase, e.g., adenine, guanine, cytosine, 5-methylcytosine, or uracil.

Another modification of the NAP molecule involves chemically linking to the NAP one or more ligands, moieties or conjugates that enhance the activity, cellular distribution or cellular uptake of the NAP molecule. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4: 1053-1060), a thioether, e.g., beryl-S-tritylthiol (Manoharan et ah, Ann. N.Y. Acad. Sci., 1992, 660:306-309; Manoharan et al, Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10: 1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

The NAP molecule may be optionally conjugated with a ligand at either the 3′ and/or 5′ end. Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin, N-acetylgalactosamine, or hyaluronic acid); or a lipid. The ligand can also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucoseamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B 12, vitamin A, biotin, or an RGD peptide or RGD peptide mimetic.

Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g., cholesterol, cholic acid, adamantane acetic acid, 1-pyrene butyric acid, dihydrotestosterone, 1,3-Bis-0(hexadecyl)glycerol, geranyloxyhexyl group, hexadecylglycerol, borneol, menthol, 1,3-propanediol, heptadecyl group, palmitic acid, myristic acid, 03-(oleoyl)lithocholic acid, 03-(oleoyl)cholenic acid, dimethoxytrityl, or phenoxazine)and peptide conjugates (e.g., antennapedia peptide, Tat peptide), alkylating agents, phosphate, amino, mercapto, PEG (e.g., PEG-40K), MPEG, [MPEG]2, polyamino, alkyl, substituted alkyl, radiolabeled markers, enzymes, haptens (e.g. biotin), transport/absorptionfacilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a hepatic cell. Ligands can also include hormones and hormone receptors. They can also include non-peptidic species, such as lipids, lectins, carbohydrates, vitamins, cofactors, multivalent lactose, multivalent galactose, N-acetyl-galactosamine, N-acetyl-gulucosamine multivalent mannose, or multivalent fucose. The ligand can be, for example, a lipopolysaccharide, an activator of p38 MAP kinase, or an activator of NF-KB.

In some embodiments, the NAP molecule described herein can be conjugated to a GalNAc derivative ligand through a bivalent or trivalent branched linker. Examples of suitable bivalent and trivalent branched linker groups conjugating GalNAc (N-acetylgalactosamine) derivatives include, but are not limited to, the structures recited above as formulas II, VII, XI, X, and XIII.

Examples of the galnac ligand can include those ligands described in WO2016077321, US20190256849, US20190211333, and US20200270611, which are incorporated herein by reference.

The ligand can be a substance, e.g., a drug, which can increase the uptake of the NAP agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin.

Ligand-conjugated oligonucleotides of the invention may be synthesized by the use of an oligonucleotide that bears a pendant reactive functionality, such as that derived from the attachment of a linking molecule onto the oligonucleotide (described below). This reactive oligonucleotide may be reacted directly with commercially-available ligands, ligands that are synthesized bearing any of a variety of protecting groups, or ligands that have a linking moiety attached thereto.

In the ligand-conjugated oligonucleotides and ligand-molecule bearing sequence-specific linked nucleosides of the present invention, the oligonucleotides and oligonucleosides may be assembled on a suitable oligonucleotide synthesizer utilizing standard nucleotide or nucleoside precursors, or nucleotide or nucleoside conjugate precursors that already bear the linking moiety, ligand-nucleotide or nucleoside-conjugate precursors that already bear the ligand molecule, or non-nucleoside ligand-bearing building blocks.

When using nucleotide-conjugate precursors that already bear a linking moiety, the synthesis of the sequence-specific linked nucleosides is typically completed, and the ligand molecule is then reacted with the linking moiety to form the ligand-conjugated oligonucleotide. In some embodiments, the oligonucleotides or linked nucleosides of the present invention are synthesized by an automated synthesizer using phosphoramidites or thiophosphoramidites derived from ligand-nucleoside conjugates in addition to the standard phosphoramidites and non-standard phosphoramidites that are commercially available and routinely used in oligonucleotide synthesis.

In some embodiments, the conjugate or ligand described herein can be attached to a NAP molecule with various linkers that can be cleavable or non-cleavable. The term “linker” or “linking group” means an organic moiety that connects two parts of a compound, e.g., covalently attaches two parts of a compound. Linkers typically comprise a direct bond or an atom such as oxygen or sulfur, a unit such as NR8, C(O), C(0)NH, SO, S02, SO2NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by O, S, S(O), S02, N(R8), C(O), substituted or unsubstituted aryl, substituted or unsubstituted heteroaryl, substituted or unsubstituted heterocyclic; where R8 is hydrogen, acyl, aliphatic or substituted aliphatic. In one embodiment, the linker is between about 1-24 atoms, 2-24, 3-24,4-24, 5-24, 6-24, 6-18, 7-18, 8-18 atoms, 7-17, 8-17, 6-16, 7-16, or 8-16 atoms.

A cleavable linking group is one which is sufficiently stable outside the cell, but which upon entry into a target cell is cleaved to release the two parts the linker is holding together. In a preferred embodiment, the cleavable linking group is cleaved at least about 10 times, 20, times, 30 times, 40 times, 50 times, 60 times, 70 times, 80 times, 90 times or more, or at least about 100 times faster in a target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing a cationic lipid from the ligand inside the cell, or into the desired compartment of the cell.

A linker can include a cleavable linking group that is cleavable by a particular enzyme. The type of cleavable linking group incorporated into a linker can depend on the cell to be targeted.

For example, a liver-targeting ligand can be linked to a cationic lipid through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

Linkers that contain peptide bonds can be used when targeting cell types rich in peptidases, such as liver cells and synoviocytes. In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue. Thus, one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It can be useful to make initial evaluations in cell-free or culture conditions and to confirm by further evaluations in whole animals. In preferred embodiments, useful candidate compounds are cleaved at least about 2, 4, 10, 20, 30, 40, 50, 60, 70, 80, 90, or about 100 times faster in the cell (or under in vitro conditions selected to mimic intracellular conditions) as compared to blood or serum (or under in vitro conditions selected to mimic extracellular conditions).

In one embodiment, a cleavable linking group is a redox cleavable linking group that is cleaved upon reduction or oxidation. In another embodiment, a cleavable linker comprises an acid cleavable linking group. In another embodiment, a cleavable linker comprises an ester-based cleavable linking group. In yet another embodiment, a cleavable linker comprises a peptide-based cleavable linking group.

In some embodiments, the NAP molecule may range from 10-50 nucleotides in length. For example, the NAP molecule may be between 10-45 nucleotides in length, 15-45 nucleotides in length, 20-45 nucleotides in length, 22-42 nucleotides in length, or 20-40 nucleotides in length.

### Compositions

The present disclosure also encompasses pharmaceutical compositions comprising NAPs of the present disclosure. One embodiment is a pharmaceutical composition comprising a NAP of the present disclosure and a pharmaceutically acceptable diluent or carrier.

In some embodiments, the pharmaceutical composition containing the NAP of the present disclosure is formulated for systemic administration via parenteral delivery. Parenteral administration includes intravenous, intra-arterial, subcutaneous, intraperitoneal or intramuscular injection or infusion; also subdermal administration, e.g., via an implanted device. In a preferred embodiment, the pharmaceutical composition containing the NAP of the present disclosure is formulated for subcutaneous (SC) or intravenous (IV) delivery. Formulations for parenteral administration may include sterile aqueous solutions, which may also contain buffers, diluents and other pharmaceutically acceptable additives as understood by the skilled artisan. For intravenous use, the total concentration of solutes may be controlled to render the preparation isotonic.

In some embodiments, the NAPs of the present disclosure are in the form of a chelate complex comprising two or more NAPs linked intermolecularly by a divalent or multivalent metal cation. NAP chelate complexes may contain, e.g., multivalent metal cations such as calcium, magnesium, cobalt, iron, manganese, barium, nickel, copper, zinc, cadmium, mercury and lead.

The pharmaceutical compositions containing the NAP of the present disclosure are useful for treating a disease or disorder, e.g., associated with the expression or activity of an HBV gene.

### Methods of Use

The following discussion is presented by way of example only, and is not intended to be limiting.

One aspect of the present technology includes methods for treating a subject diagnosed as having, suspected as having, or at risk of having a viral infection comprising administering to the subject in need thereof a therapeutically effective amount of the NAP of the present disclosure.

Some embodiments include methods for treating a subject diagnosed as having, suspected as having, or at risk of having an HBV infection and/or an HBV-associated disorder. In therapeutic applications, compositions comprising the NAP of the present technology are administered to a subject suspected of, or already suffering from such a disease (such as, e.g., presence of an such as HBV antigen surface and envelope antigens (e.g., HBsAg and/or HBeAg) in the serum and/or liver of the subject, or elevated HBV DNA or HBV viral load levels), in an amount sufficient to cure, or at least partially arrest, the symptoms of the disease, including its complications and intermediate pathological phenotypes in development of the disease.

Subjects suffering from an HBV infection and/or an HBV-associated disorder can be identified by any or a combination of diagnostic or prognostic assays known in the art. For example, typical symptoms of HBV infection and/or an HBV-associated disorder include, but are not limited to the presence of serum and/or liver HBV antigen (e.g., HBsAg and/or HBeAg), elevated ALT, elevated AST, the absence or low level of anti-HBV antibodies, liver injury, cirrhosis, delta hepatitis, acute hepatitis B, acute fulminant hepatitis B, chronic hepatitis B, liver fibrosis, end-stage liver disease, hepatocellular carcinoma, serum sickness-like syndrome, anorexia, nausea, vomiting, low-grade fever, myalgia, fatigability, disordered gustatory acuity and smell sensations (aversion to food and cigarettes), right upper quadrant and epigastric pain (intermittent, mild to moderate), hepatic encephalopathy, somnolence, disturbances in sleep pattern, mental confusion, coma, ascites, gastrointestinal bleeding, coagulopathy, jaundice, hepatomegaly (mildly enlarged, soft liver), splenomegaly, palmar erythema, spider nevi, muscle wasting, spider angiomas, vasculitis, variceal bleeding, peripheral edema, gynecomastia, testicular atrophy, abdominal collateral veins (caput medusa), high levels of alanine aminotransferase (ALT) and aspartate aminotransferase (AST) (within a range of 1000-2000 IU/mL), ALT levels higher than AST levels, elevated gamma-glutamyl transpeptidase (GGT) and/or alkaline phosphatase (ALP) levels, decreased albumin levels, elevated serum iron levels, leukopenia (i.e., granulocytopenia), lymphocytosis, increased erythrocyte sedimentation rate (ESR), shortened red blood cell survival, hemolysis, thrombocytopenia, a prolongation of the international normalized ratio (INR), the presence of serum HBV DNA, elevation of the aminotransferases (<5 times the ULN), increased bilirubin levels, prolonged prothrombin time (PT), hyperglobulinemia, the presence of tissue-nonspecific antibodies, such as anti-smooth muscle antibodies (ASMAs) or antinuclear antibodies (ANAs), the presence of tissue-specific antibodies, such as antibodies against the thyroid gland, elevated levels of rheumatoid factor (RF), hyperbilirubinemia, low platelet and white blood cell counts, AST levels higher than ALT levels, lobular inflammation accompanied by degenerative and regenerative hepatocellular changes, and predominantly centrilobular necrosis.

In some embodiments, subjects treated with the NAP composition of the present technology will show amelioration or elimination of one or more of the following conditions or symptoms: the presence of serum and/or liver HBV antigen (e.g., HBsAg and/or HBeAg), the absence or low level of anti-HBV antibodies, liver injury, cirrhosis, delta hepatitis, acute hepatitis B, acute fulminant hepatitis B, chronic hepatitis B, liver fibrosis, end-stage liver disease, hepatocellular carcinoma, serum sickness-like syndrome, anorexia, nausea, vomiting, low-grade fever, myalgia, fatigability, disordered gustatory acuity and smell sensations (aversion to food and cigarettes), right upper quadrant and epigastric pain (intermittent, mild to moderate), hepatic encephalopathy, somnolence, disturbances in sleep pattern, mental confusion, coma, ascites, gastrointestinal bleeding, coagulopathy, jaundice, hepatomegaly (mildly enlarged, soft liver), splenomegaly, palmar erythema, spider nevi, muscle wasting, spider angiomas, vasculitis, variceal bleeding, peripheral edema, gynecomastia, testicular atrophy, abdominal collateral veins (caput medusa), ALT levels higher than AST levels, leukopenia (i.e., granulocytopenia), decreased albumin levels, elevated serum iron levels, lymphocytosis, increased erythrocyte sedimentation rate (ESR), shortened red blood cell survival, hemolysis, thrombocytopenia, a prolongation of the international normalized ratio (INR), the presence of serum HBV DNA, prolonged prothrombin time (PT), hyperglobulinemia, the presence of tissue-nonspecific antibodies, such as anti-smooth muscle antibodies (ASMAs) or antinuclear antibodies (ANAs), the presence of tissue-specific antibodies, such as antibodies against the thyroid gland, hyperbilirubinemia, low platelet and white blood cell counts, AST levels higher than ALT levels, lobular inflammation accompanied by degenerative and regenerative hepatocellular changes, and predominantly centrilobular necrosis.

In some embodiments, subjects treated with the NAP composition of the present technology will show a reduction in the expression levels of one or more biomarkers selected from among alanine aminotransferase (ALT), aspartate aminotransferase (AST), gamma-glutamyl transpeptidase (GGT), alkaline phosphatase (ALP), bilirubin, and rheumatoid factor (RF), compared to untreated subjects suffering from an HBV infection and/or an HBV-associated disorder.

The present disclosure provides a method for treating a subject diagnosed as having, or suspected as having an HBV infection and/or an HBV-associated disorder comprising administering to the subject an effective amount of a NAP composition of the present technology.

The NAPs and compositions of the present disclosure may be used in combination with, e.g., a non-nucleotide antiviral polymer, an antisense molecule, an siRNA, or a small molecule drug. For example, the NAP may be administered with an oligonucleotide containing a nucleobase sequence that is complementary or hybridizes to a target nucleic acid sequence of a known viral DNA or RNA sequence, for example, in HBV.

The disclosed NAPs may be administered alone or in combination with one or more additional treatments for the targeted ailment, e.g., at least one other antiviral drug in combination with the NAP. In some embodiments, the disclosed NAPs may be administered alone or in combination with one or more additional treatments for HBV infection. In combination therapies, it is understood that the NAPs and one or more additional treatments for HBV infection may be administered simultaneously in the same or separate compositions, or administered separately, at the same time or sequentially.

In some embodiments, the disclosed NAPs are administered in combination with HBV replication inhibitors or immune modulator agents or in regimens that combine anti-HBV oligonucleotide agents with both HBV replication inhibitors and immune modulation agents. In embodiments, the disclosed oligonucleotide constructs are administered in combination with standard of care treatment for HBV infection. Standard of care treatment for HBV infection can include inhibitors of viral polymerase such as nucleotide/nucleotide analogs (e.g., Lamivudine, Telbivudine, Entecavir, Adefovir, Tenofovir, and Clevudine, Tenofovir alafenamide (TAF), CMX157, and AGX-1009) and Interferons (e.g., Peg-IFN-2a and IFN-a-2b, Interferon lambda). In embodiments, the disclosed NAPs are administered in combination with one or more oligonucleotides after either simultaneous (co-administration) or sequential dosing. Oligonucleotides can include siRNA such as ALN-HBV, ARB-1467, ARC-520 and ARC-521, antisense oligonucleotides such as RG6004 (LNA HBV), Ionis-HBV_{Rx} and Ionis-HBV-L_{Rx}, miRNA mimics or inhibitors, aptamers, steric blockers, saRNA, shRNA, immunomodulatory and/or other HBsAg release inhibiting such as REP 2139 and REP 2165 oligonucleotides. In embodiments, the disclosed NAPs are administered in combination with one or more antiviral agents such as viral replication inhibitors. In embodiments, the disclosed NAPs are administered in combination with HBV Capsid inhibitors. HBV capsid inhibitors can include NVR 3-778, AB-423, GLS-4, Bayer 41-4109, HAP-1, and AT-1. In embodiments, the disclosed NAPs are administered in combination with one or more immunomodulators such as TLR agonists. TLR agonists can include GS-9620, ARB-1598, ANA975, RG7795(ANA773), MEDI9197, PF-3512676, and IMO-2055. In embodiments, the disclosed NAPs are administered in combination with HBV vaccines. HBV vaccines can include Heplislav, ABX203, and INO-1800.

For therapeutic applications, a NAP composition of the present disclosure is administered to the subject. In some embodiments, the NAP composition is administered one, two, three, four, or five times per day. In some embodiments, the NAP composition is administered more than five times per day. Additionally or alternatively, in some embodiments, the NAP composition is administered every day, every other day, every third day, every fourth day, every fifth day, or every sixth day. In some embodiments, the NAP composition is administered weekly, bi-weekly, tri-weekly, or monthly. In some embodiments, the NAP composition is administered for a period of one, two, three, four, or five weeks. In some embodiments, the NAP composition is administered for six weeks or more. In some embodiments, the NAP composition is administered for twelve weeks or more. In some embodiments, the NAP composition is administered for a period of less than one year. In some embodiments, the NAP composition is administered for a period of more than one year.

In some embodiments of the methods of the present disclosure, the NAP composition is administered daily for 1 week or more. In some embodiments of the methods of the present disclosure, the NAP composition is administered daily for 2 weeks or more. In some embodiments of the methods of the present disclosure, the NAP composition is administered daily for 3 weeks or more. In some embodiments of the methods of the present disclosure, the NAP composition is administered daily for 4 weeks or more. In some embodiments of the methods of the present disclosure, the NAP composition is administered daily for 6 weeks or more. In some embodiments of the methods of the present disclosure, the NAP composition is administered daily for 12 weeks or more.

### Definitions

It is to be understood that the terminology used herein is for the purpose of describing particular embodiments only and is not intended to limit the scope of the present invention. The following definitions shall apply unless otherwise indicated.

“Pharmaceutically acceptable” refers to a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. and Drug administration.

“Modified nucleoside” refers to a nucleoside having, independently, a modified sugar moiety and/or modified nucleobase. It is understood that nucleosides can be linked through intersubunit linkages, such as phosphodiester intersubunit linkages, thiophosphate intersubunit linkages, phosphoramidate intersubunit linkages, and thiophosphoramidate intersubunit linkages “Modified nucleotides” may refer to a nucleoside and intersubunit linkage together.

The term “nucleic acid polymer” or “NAP” refers to a single stranded oligonucleotide that does not contain sequence specific functionality related to hybridization with a nucleic acid target or forming a sequence specific secondary structure that results in binding to a specific protein. The biochemical activity of a NAP does not dependent on Toll-like receptor recognition of oligonucleotides, hybridization with a target nucleic acid or aptameric interaction requiring a specific secondary/tertiary oligonucleotide structure derived from a specific order of nucleotides present. NAPs can include base and or linkage and or sugar modifications as described herein.

The term “NAP chelate complex” refers to a complex of two or more NAPs in solution linked intermolecularly by a divalent metal cation as described in US 2012/61695040 and US 2012/0046348.

The term “virus” as it related to a subject is intended to include, without limitation, human and/or animal DNA and RNA viruses in general. DNA viruses include, for example, parvoviridae, papovaviridae, adenoviridae, herpesviridae (e.g., EBV, HSV-1, HSV-2, CMV, VZV, HHV-6, HHV-7, or HHV-8), poxviridae, hepadnaviridae (e.g., HBV, HCV, HDV), and papillomaviridae. RNA viruses include, for example, arenaviridae, bunyaviridae, calciviridae, coronaviridae, filoviridae, flaviridae, orthomyxoviridae, paramyxoviridae, picornaviridae, reoviridae, rhabdoviridae, retroviridae, or togaviridae.

“Unmodified” or “natural” nucleobases include the purine bases adenine (A) and guanine (G), and the pyrimidine bases thymine (T), cytosine (C) and uracil (U). “Modified nucleobases” include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-am-oelhoxy)-H-pyrimido[5,4-b][1,4]benzoxazin-2(3H)-one), carbazole cytidine (2H-pyrimido[4,5-b]indol-2-one), pyridoindole cytidine (H-pyrido[3,2,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine, and 2-pyridone. In some embodiments, the modified nucleobase is selected from the group consisting of 5-methylcytosine, 2,6-diaminopurine, and 5-methyluracil.

“Subject” refers to mammals and includes humans and non-human mammals. In some embodiments, the subject is a human, such as an adult human.

“Treating” or “treatment” of a disease in a subject refers to (1) preventing the disease from occurring in a subject that is predisposed or does not yet display symptoms of the disease; (2) inhibiting the disease or arresting its development; or (3) ameliorating or causing regression of the disease.

“Therapeutically effective amount” means an amount of a pharmaceutical agent that provides a therapeutic benefit to a subject.

“Pharmaceutically acceptable salt” means physiologically and pharmaceutically acceptable salts of the compounds of the present disclosure, i.e., salts that retain the desired biological activity of the parent oligonucleotide/compound and do not impart undesired toxicological effects thereto.

The following abbreviations are used in this disclosure. 2′-H (deoxyribose) nucleosides are referred to by an uppercase letter corresponding to the nucleobase, e.g., A, C, G, and T. 2′-OH (ribose) nucleosides are referred to by a lowercase r and an uppercase letter corresponding to the nucleobase, e.g., rA, rC, rG, and rU. 2′-O-Me nucleosides are referred to by a lowercase m and an uppercase letter corresponding to the nucleobase, e.g., mA, mC, mG, mU, (5m)mC (2′-O-Me 5-methylcytosine). 2′-MOE nucleosides are referred to by a lowercase “moe” and an uppercase letter corresponding to the nucleobase, e.g., moeA, moeC, moeG and moeU. 2′-ribo-F nucleosides are referred to by a lowercase “f” and an uppercase letter corresponding to the nucleobase, e.g., fA, fC, fG and fU. 2′-arabino-F nucleosides are referred to by a lowercase “af” and an uppercase letter corresponding to the nucleobase, e.g., afA, afC, afG and afU.

For the backbone or intersubunit linkages of the nucleotides, phosphodiester intersubunit linkages are referred to as “po” or are generally not included in sequence details; thiophosphate intersubunit linkages are abbreviated as lowercase “ps”; phosphoramidate intersubunit linkages are abbreviated as lowercase “np”; and thiophosphoramidate intersubunit linkages are abbreviated as lowercase “nps.” It will be understood that np and nps intersubunit linkages contain a N3→P5′ linkage, which refers to nucleotides having intersubunit linkages where the 3′ moiety contains N (e.g., NH) and is linked through a P. For example, the following structure has a N3→P5′ linkage:

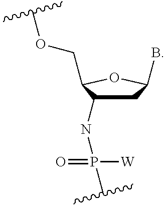

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely”, “only” and the like in connection with the recitation of claim elements, or use of a “negative” limitation.

The term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term. Certain ranges are presented herein with numerical values being preceded by the term “about”. The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context in which it is presented, provides the substantial equivalent of the specifically recited number.

It is also to be appreciated that the various modes of treatment or prevention of the diseases or conditions described herein are intended to mean “substantial,” which includes total but also less than total treatment or prevention, and wherein some biologically or medically relevant result is achieved. The treatment may be a continuous prolonged treatment for a chronic disease or a single, or few time administrations for the treatment of an acute condition.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

This disclosure is not limited to particular embodiments described, as such may vary. It is also to be understood that the terminology used herein is for the purpose of describing particular embodiments only, and is not intended to be limiting, since the scope of the present invention will be limited only by the appended claims.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order that is logically possible.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention.

Further, the dates of publication provided may be different from the actual publication dates that may need to be independently confirmed.

### EXAMPLES

The following examples illustrate certain embodiments of the present disclosure to aid the skilled person in practicing the disclosure. Accordingly, the examples are in no way considered to limit the scope of the disclosure.

### Methods of Making

All the monomers are dried in vacuum desiccator with desiccants (KOH and P205, RT 24 h). Synthesis solid supports (CPG) attached to the first 5′ residue are obtained from commercially available sources. All other synthesis reagents and solvents are obtained from commercially available sources and used as such. The chemicals and solvents for post synthesis workflow are purchased from commercially available sources and used without any purification or treatment. Solvent (Acetonitrile) and solutions (amidite and activator) are stored over molecular sieves during synthesis.

The NAPs are synthesized on an ABI-394 synthesizer using the standard 93-step cycle written by the manufacturer. The solid support is controlled pore glass and the monomers contained standard protecting groups. Each oligonucleotide is individually synthesized using commercially available 5′-O-(4,4′-dimethoxytrityl)-3′-O-(2-cyanoethyl-N,N-diisopropyl) DNA and or 2′-O-Me phosphoramidite monomers of 6-N-benzoyladenosine (A^{Bz}), 4-N-acetylcytidine (C^{Ac}), 2-N-isobutyrylguanosine (GU), and Thymidine (T), according to standard solid phase oligonucleotide synthesis protocols. The phosphoramidites are purchased from commercially available sources. The 2′-O-Me-2,6,diaminopurine phosphoramidite is purchased from commercially available sources. The DDTT ((dimethylamino-methylidene) amino)-3H-1,2,4-dithiazaoline-3-thione is used as the sulfur-transfer agent for the synthesis of oligoribonucleotide phosphorothioates. Modified oligonucleotides are obtained using an extended coupling of 0.1M solution of phosphoramidite in CH₃CN in the presence of 5-(ethylthio)-1H-tetrazole activator to a solid bound oligonucleotide followed by standard capping, oxidation and deprotection. The stepwise coupling efficiency of all modified phosphoramidites is more than 98%. Oligonucleotide-bearing solid supports are heated with aqueous ammonia/ethanol (3:1) solution at 55° C. for 8 h to deprotect the base labile protecting groups.

### Synthesis of Phosphoramidate (NP) and Thiophosphoramidate (NPS) Modified Oligonucleotides

The NP and NPS modified oligonucleotides are synthesized on an ABI-394 synthesizer using the 93-step cycle written with modifications to deblock, coupling and wait steps. The solid support is 3′-NHTr-5′-LCAA-CPG. Each oligonucleotide is individually synthesized using 3′-NH-Tr-5′-O-(2-cyanoethyl-N,N-diisopropyl) DNA phosphoramidite monomers of 6-N-benzoyladenosine (A^{Bz}), 4-N-Benzylcytidine (C^{Bz}), 2-N-isobutyrylguanosine (G^{iBu}), and Thymidine (T), according to standard solid phase phosphoramidite chemistry protocols by using the procedure described in Nucleic Acids Research, 1995, Vol. 23, No. 14 2661-2668.

### 2′-OMe-3′-NHTr Building Blocks for Oligomer Synthesis

The 2′-O-Me 3′-NH-MMTr-5′-O-(2-cyanoethyl-N,N-diisopropyl) phosphoramidite monomers of 6-N-benzoyladenosine (A^{Bz}), 4-N-Benzylcytidine (C^{Bz}), 2-N-isobutyrylguanosine (G^{iBu}), and Uridine (U) as shown below are synthesized using the procedure described in WO 200118015 A1

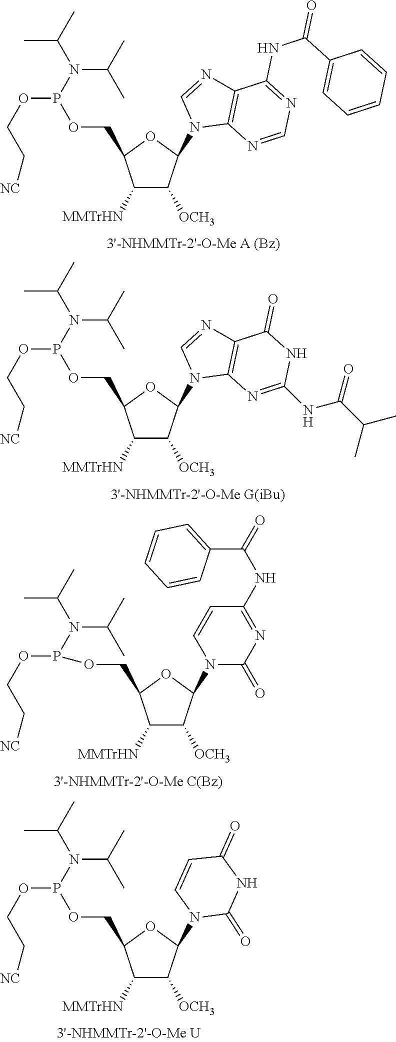

### 2′-O-Me-3′-NHTr Building Blocks for Oligomer Synthesis

Exemplary phosphoroamidates include:

Raw material description

3′-NHTr-dA(Bz)
3′-NHTr-dC(Bz)
3′-NHTr-dG(iBu)
3′-NHTr-T:
3′-NHMMTr-2′-F-A(NH-Bz)
3′-NHMMTr-2′-F-C(NH-Bz)
3′-NHMMTr-2′-F-G(NH-iBu)
3′-NHMMTr-2′-F-U:
3′-NHMMTr-2′-OMe-A(NH-Bz)
3′-NHMMTr-2′-OMe-C(NH-Bz)
3′-NHMMTr-2′-OMe-G(NH-iBu)
3′-NHMMTr-2′-OMe U:
3′-NHTr (dA, dC, dG and dT)-CPG 500 Å:
Loading: 64-83 μmol/g

The reverse phosphoramidite 3′-O-DMT-deoxy Adenosine (NH-Bz), 5′-O-(2-cyanoethyl-N,N-diisopropyl phosphoramidite, 3′-O-DMT-deoxy Guanonosine (NH-ibu), 5′-O-(2-cyanoethyl-N,N-diisopropyl phosphoramidite, 3′-O-DMT-deoxy Cytosine (NH-Bz), 5′-O-(2-cyanoethyl-N,N-diisopropyl phosphoramidite, 3′-O-DMT-deoxy Thymidine (NH-Bz), 5′-O-(2-cyanoethyl-N,N-diisopropyl phosphoramidite and reverse solid supports are purchased from commercially-available sources (Chemgenes).

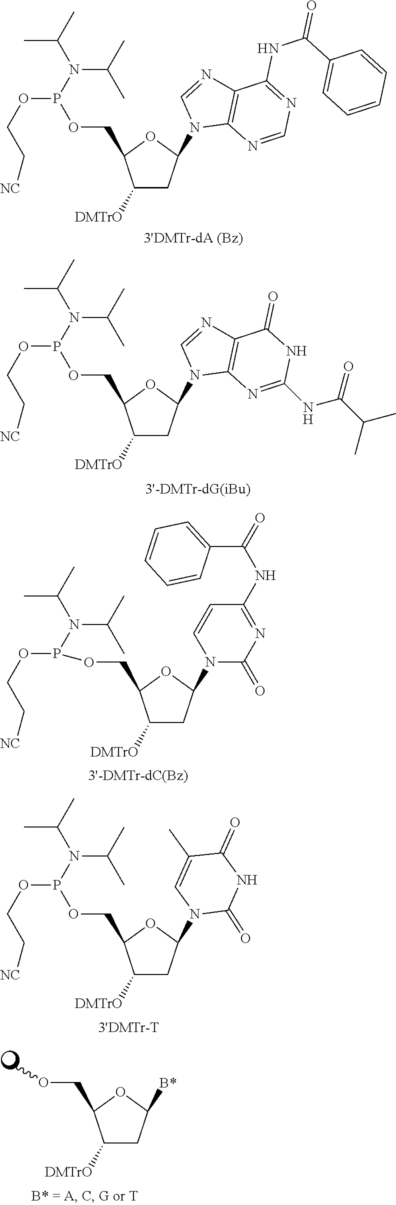

### Reverse DNA Building Blocks for Oligomer Synthesis

Exemplary reverse phosphoroamidites used for this disclosure include:

Raw material description

3′-O-DMTr-2′-OMe-A(NH-Bz)
3′-O-DMTr-2′-OMe-C(NH-Bz)
3′-O-DMTr-2′-OMe-G(NH-iBu)
3′-O-DMTr-2′-OMe-U:
3′-ODMTr (dA, dC, dG and dT)-CPG 500 Å:
Loading: 64-83 μmol/g

For making the oligomers with the following modifications: 2′—F—NPS—PS-2′-F—NPS; 2′-F—NP—PS-2′-F—NP; 2′-OMe-NP—PS-2′-OMe-NP; 2′-OMe-NPS-DNA-PS-2′-OMe-NPS, the synthesis is carried out on a 1 μM scale in a 5′ to 3′ direction with the 5′-phosphoramidite monomers diluted to a concentration of 0.1 M in anhydrous CH₃CN in the presence of 5-(benzylthio)-1H-tetrazole activator (coupling time 2.0-4.0 min) to a solid bound oligonucleotide followed by standard capping, oxidation and deprotection afforded modified oligonucleotides. The stepwise coupling efficiency of all modified phosphoramidites is more than 98%. The DDTT (dimethylamino-methylidene) amino)-3H-1, 2, 4-dithiazaoline-3-thione is used as the sulfur-transfer agent for the synthesis of oligoribonucleotide phosphorothioates. Oligonucleotide-bearing solid supports are heated at room temperature with aqueous ammonia/Methylamine (1:1) solution for 3 h in shaker to cleavage from support and deprotect the base labile protecting groups.

### Examples 1-4

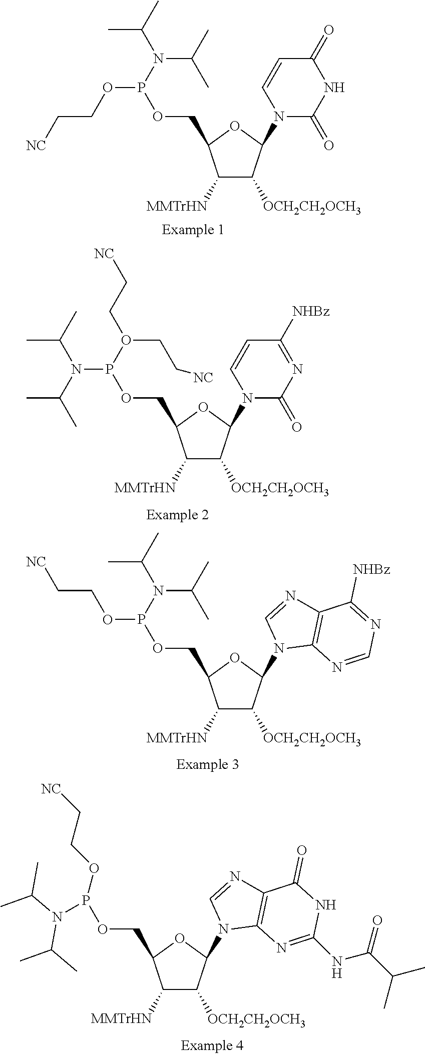

The appropriately protected 2′-O-methoxy ethyl-3′-aminonucleoside-5′-phosphoramidite building blocks (examples 1-4 are prepared after chemical transformations shown in Schemes 1-4.

First for synthesis of uracil based 3′-NH-MMTr-2′-O-methoxyethyl phosphoramidites example 5, key 3′-azido-2′-methoxyethyl intermediate 3 is obtained in low yields via an-hydro intermediate 2 as shown in scheme 1.

Due to low yielding alkylation, 3-1 is reacted with BOMCl/DBU to give N-3 protected intermediate 3-4, which is alkylated by using 2-bromoethyl methyl ether/Ag₂O/NaI/DMF to give 2′-O-methoxyethyl derivative 3-5 as shown below in scheme 1. Deprotection of N-3-BOM group using hydrogenation condition (Pd/C/H₂) resulted in 10-20% desired 3′-amino intermediate3-6a along with significant over reduced side product 3-6b.

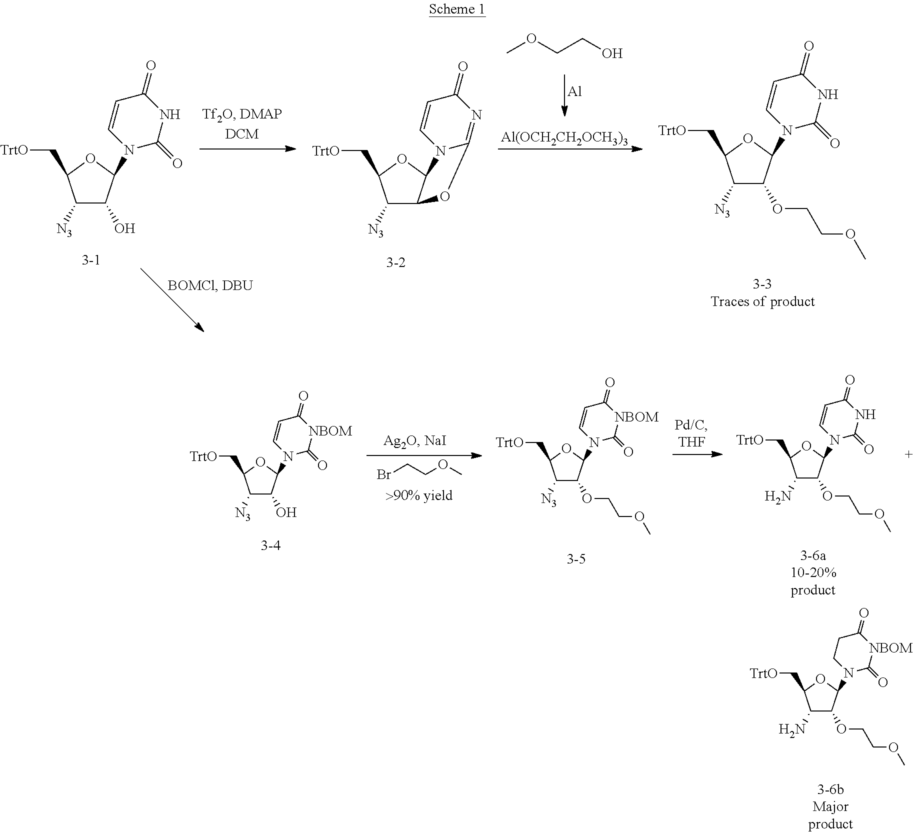

2′-O-alkylation in high yield is obtained as shown below in scheme 2. For this purpose, 3-1 is treated with PMBCl/DBU/DMF to give N-3 protected intermediate 4-2, which is subjected for 2′-O alkylation using 2-bromoethyl methyl ether/Ag₂O/NaI/DMF to give 2′-O-methoxyethyl derivative 4-3. Then, 5′-de-tritylation of 4-3 and re-protection of 5′-hydroxyl group using benzoyl chloride afforded 4-5.

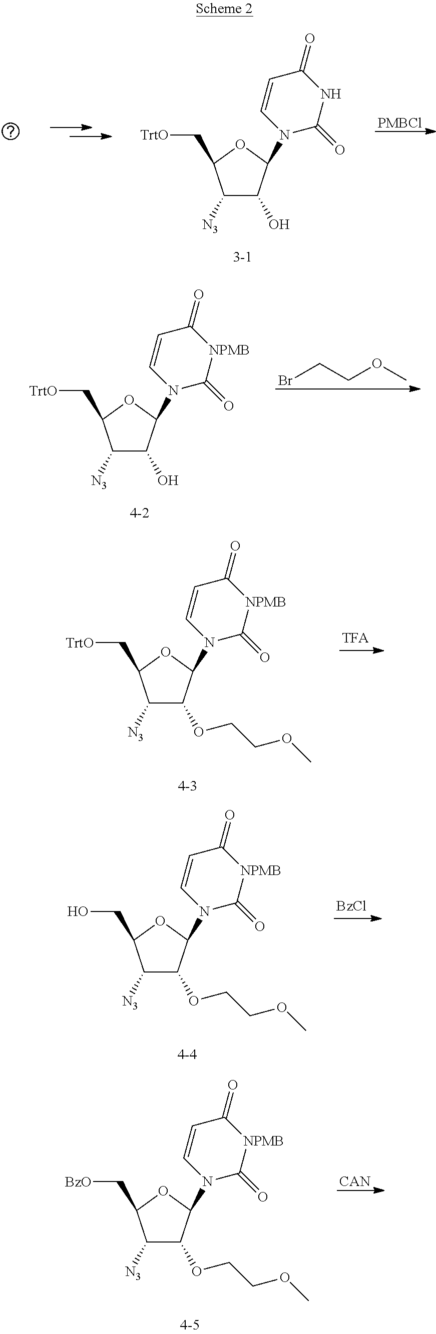

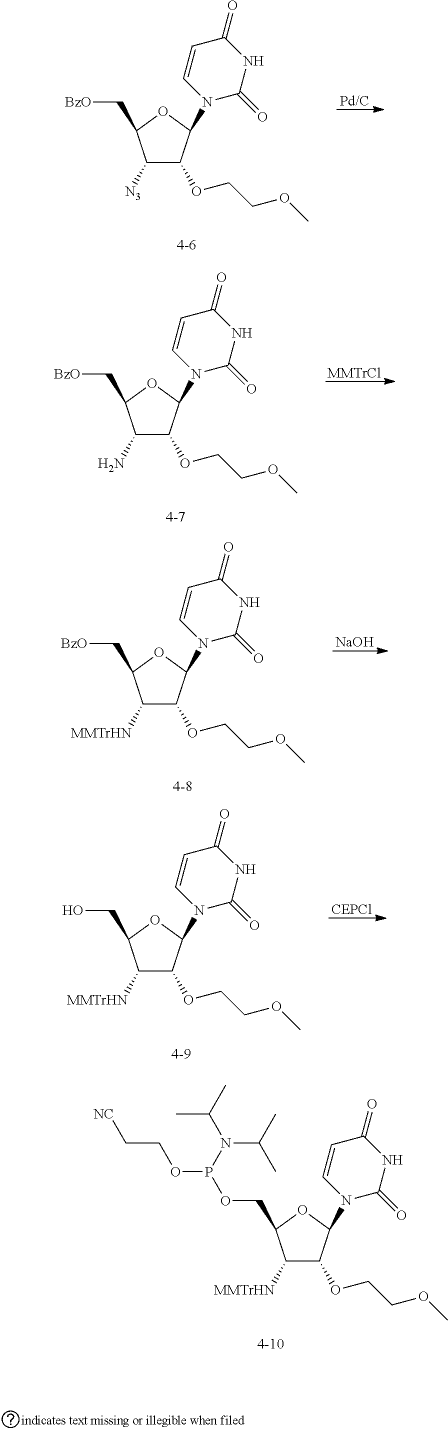

De-protection of PMB group of intermediate 4-5 in mild conditions gives 4-6. 3′-Azido group of intermediate 4-6 is reduced to an amine, which is then immediately protected, such as reaction with 4-monomethoxytritylchloride, to give 4-8. The 5′-benzyl ester is then cleaved using an alkaline solution, followed by phosphitylation using known protocols to give the desired 2′-O-methoxyethoxy uridine phosphoramidite monomer 4-10.

Preparation of (4-2): To a solution of 3-1 (45.30 g, 88.56 mmol) in DMF (120.00 mL) is added PMBCl (20.80 g, 132.84 mmol) and DBU (44.61 g, 177.12 mmol), the mixture is stirred at r.t. for 2 h. Water is added, extracted with EA. The organic layer is concentrated and purified by column to give 4-2 (52.00 g, 82.32 mmol) as a white solid. ESI-LCMS: m/z 632.3 [M+H]⁺.

Preparation of (4-3): To a solution of 4-2 (50.00 g, 79.15 mmol) in DMF (120.00 mL) is added 2-Bromoethyl methyl ether (16.50 g, 118.73 mmol) and Ag₂O (18.34 g, 79.15 mmol, 2.57 mL), then NaI (5.93 g, 39.58 mmol) is added. The reaction mixture is stirred at r.t. for 12 h. LC-MS showed work well. Filtered and added water and EA, the organic layer is concentrated and purified by column to give 4-3 (52.00 g, 75.39 mmol) as a colorless oil. ESI-LCMS: m/z 690.4 [M+H]⁺.

Preparation of (4-4): To a solution of 4-3 (52.00 g, 75.39 mmol) in DCM (200.00 mL) is added TFA (150.00 mL). The mixture is stirred at r.t. for 1 h. The reaction mixture is slowly added to cold NH₄OH, extracted with DCM. The organic layer is concentrated and purified to give 4-4 (31.00 g, 69.28 mmol) as a colorless oil. ESI-LCMS: m/z 448.2 [M+H]⁺. ¹H-NMR (DMSO-d₆, 400 MHz): δ ppm 8.02 (d, J=8.12 Hz, 1H), 7.26-7.23 (m, 2H), 6.87-6.84 (m, 2H), 5.87-5.81 (m, 2H), 5.38 (t, J=5.0 Hz, 1H), 4.96-4.85 (m, 2H), 4.36-4.34 (m, 1H), 4.17-4.14 (m, 1H), 4.00-3.97 (m, 1H), 3.83-3.77 (m, 1H), 3.75-3.72 (m, 1H), 3.71 (s, 3H), 3.70-3.68 (m, 1H), 3.61-3.56 (m, 1H), 3.45-3.43 (m, 2H), 3.18 (s, 3H).

Preparation of (4-5): To a solution of 4-4 (31.00 g, 69.28 mmol) in Pyridine (200.00 mL) is added BzCl (13.14 g, 93.87 mmol), the reaction mixture is stirred at r.t. for 15 min and concentrated and purified by column to give 4-5 (35.10 g, 63.8 mmol) as a white solid. ESI-LCMS: m/z 552.2 [M+H]⁺.

Preparation of (4-6): To a solution of 4-5 (35.10 g, 63.8 mmol) in acetonitrile (300.00 mL) and water (100.00 mL) is added Ceric ammonium nitrate (105 g, 191.40 mmol), the reaction mixture is stirred at r.t. for 12 h and concentrated and extracted with EA. The organic layer is concentrated and purified by column to give 4-6 (27.5 g, 63.75 mmol) as a yellow solid. ESI-LCMS: m/z 432.2 [M+H]⁺.

Preparation of (4-7): To a solution of 4-6 (27.50 g, 63.75 mmol) in THF (500.00 mL) is added Pd/C (3.00 g), the reaction mixture is stirred at r.t. for 12 h and filtered and concentrated to give 4-7 (25.00 g, 61.67 mmol) as a yellow solid. ESI-LCMS: m/z 406.2 [M+H]⁺.

Preparation of (4-8): To a solution of 4-7 (25.00 g, 61.67 mmol) in DCM (300.00 mL) is added MMTrCl (28.49 g, 92.51 mmol) and Collidine (14.95 g, 123.34 mmol), then AgNO₃ (15.7 g, 92.5 mmol) is added. The reaction mixture is stirred at r.t. for 1 h., and filtered and the organic layer is washed water, dried over Na₂SO₄ and purified by silica gel column to give 4-8 (33.00 g, 48.69 mmol) as a yellow solid.

Preparation of (4-9): To a solution of 4-8 (14.50 g, 21.39 mmol) is added 1 N NaOH in methanol (200 mL) in water (20 mL), the reaction mixture is stirred at r.t. for 1 h. and concentrated and extracted with DCM, the organic layer is concentrated and purified by silica gel column to give 4-9 (11.50 g, 20.05 mmol) as a white solid. ¹H-NMR (DMSO-d₆, 400 MHz): δ ppm 11.26 (s, 1H), 7.95 (d, J=8.4 Hz, 1H), 7.47-7.44 (m, 4H), 7.34-7.17 (m, 8H), 6.82 (d, J=8.8 Hz, 2H), 5.50-5.48 (m, 2H), 5.13 (t, J=3.6 Hz, 1H), 4.05-3.98 (m, 3H), 3.78 (s, 3H), 3.52-3.49 (m, 1H), 3.34-3.32 (m, 2H), 3.14 (s, 3H), 3.08-3.04 (m, 1H), 2.89-2.86 (m, 1H), 2.70 (d, J=10.0 Hz, 1H), 1.51 (d, J=4.4 Hz, 1H).

Preparation of (4-10): To a solution of 4-9 (11.50 g, 20.05 mmol) in DCM (100.00 mL) is added DMAP (489.85 mg, 4.01 mmol) and DIPEA (10.36 g, 80.19 mmol, 14.01 mL). Then CEPCl (5.70 g, 24.06 mmol) is added to the solution. The mixture is stirred at r.t. for 30 min. The reaction is quenched with saturated NaHCO₃. The organic layer is washed with brine, dried over Na₂SO₄, concentrated to give the crude product. The crude product is purified by Flash-Prep-HPLC. The product is dissolved in anhydrous toluene and concentrated for three times. Then the product is dissolved anhydrous acetonitrile and concentrated for three times. This resulted in 13 g to give 4-10 as a white solid. MS m/z [M−H]⁻ (ESI): 772.3; ¹H-NMR (CDCl₃, 400 MHz): 9.01 (s, 1H), 8.07-7.61 (m, 1H), 7.53-7.41 (m, 6H), 7.29-7.15 (m, 5H), 6.79-6.76 (m, 2H), 5.63-5.57 (m, 2H), 4.27-4.15 (m, 2H), 4.06-3.95 (m, 1H), 3.85-3.77 (m, 1H), 3.75 (s, 3H), 3.69-3.35 (m, 7H), 3.23 (d, J=4 Hz, 1H), 2.26-2.91 (m, 3H), 2.59 (t, J=6.4 Hz, 1H), 1.75-1.39 (m, 1H), 1.21-1.11 (m, 12H). ³¹PNMR (162 MHz, CDCl₃): 149.10, 148.26.

### Example 5

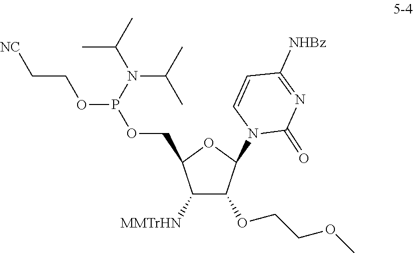

The 2′-O-methoxyethoxy-NH-benzoyl-cytosine phosphoramidite compound 5-4 is obtained by conversion of uridine intermediate 4-8 into 3′-amino cytidine analogue 5-1 followed by phosphitylation using known protocols to give the desired 2′-O-methoxyethoxy cytidine phosphoramidite monomer 5-4 as shown below in scheme 3.

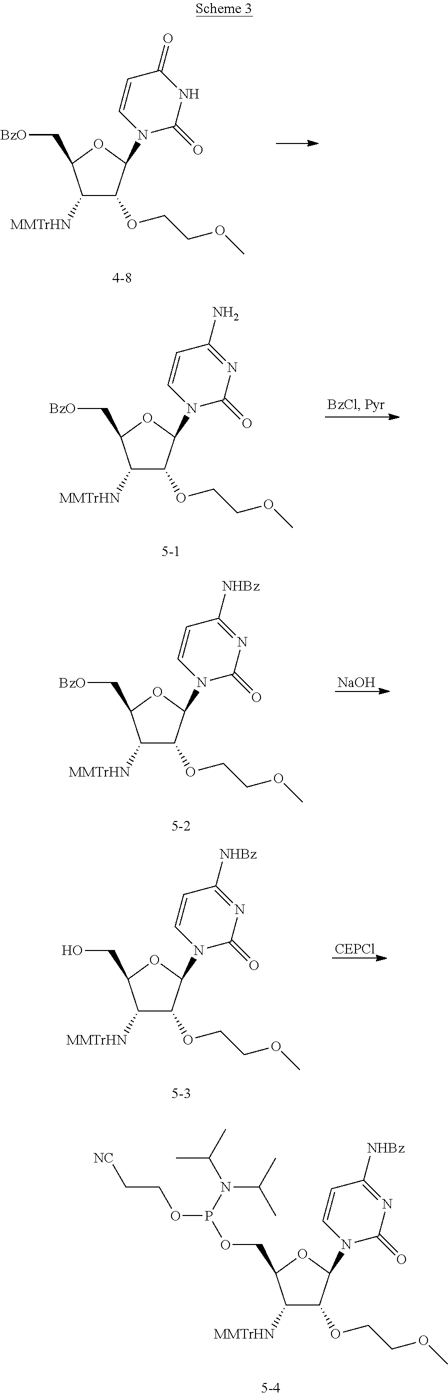

Preparation of (5-1): To a solution of 4-8 (18.50 g, 27.30 mmol) in acetonitrile (250.00 mL) is added TPSCl (16.49 g, 54.60 mmol) and DMAP (6.67 g, 54.60 mmol), then TEA (5.52 g, 54.60 mmol, 7.56 mL) is added to the solution. The reaction mixture is stirred at r.t. for 5 h under N₂. NH₄OH (50.00 mL) is added to the reaction mixture. The mixture is stirred at r.t. for 12 h. The solution is concentrated and extracted with EA. The organic layer is washed by brine and dried over Na₂SO₄. The organic layer is concentrated and purified by silica gel column to give 5-1 (16.00 g, 23.64 mmol) as a yellow solid.

Preparation of (5-2): To a solution of 5-1 (16.00 g, 23.64 mmol) in Pyridine (100.00 mL) is added BzCl (4.96 g, 35.46 mmol) at 0° C. The mixture is stirred at r.t. for 1 h. The solution is concentrated and purified by silica gel column to give 5-2 (17.40 g, 22.28 mmol) as a white solid.

Preparation of (5-3): Compound 5-2 (17.40 g, 22.28 mmol) is added to 180 mL of 1 N NaOH solution in Pyridine/MeOH/H₂O (65/30/5) at 0° C. The suspension is stirred at 0° C. for 15 min. The reaction mixture is quenched by addition of sat. NH₄Cl solution. The solution is extracted with EA and the combined organic layers are washed with sat. NaHCO₃ solution, brine, dried over Na₂SO₄, filtered, and concentrated. The residue is purified by column to give 5-3 (12.50 g, 18.47 mmol) as white solid. 1H-NMR (DMSO-d₆, 400 MHz): δ ppm 12.25 (s, 1H), 8.53 (d, J=7.6 Hz, 1H), 8.01 (d, J=5.2 Hz, 2H), 7.64-7.60 (m, 1H), 7.52-7.42 (m, 6H), 7.31 (d, J=8.8 Hz, 2H), 7.26-7.14 (m, 7H), 6.79 (d, J=8.8 Hz, 2H), 5.55 (s, 1H), 5.23 (t, J=3.6 Hz, 1H), 4.09-3.97 (m, 3H), 3.73 (s, 3H), 3.70-3.66 (m, 1H), 3.38-3.34 (m, 2H), 3.17 (s, 3H), 3.11-3.05 (m, 1H), 2.96-2.91 (m, 1H), 2.68 (d, J=10.8 Hz, 1H), 1.49 (d, J=4 Hz, 1H).

Preparation of (5-4): To a solution of 5-3 (12.50 g, 18.47 mmol) in DCM (100.00 mL) is added DMAP (451.30 mg, 3.69 mmol) and DIPEA (9.55 g, 73.88 mmol, 12.90 mL), then CEPCl (5.25 g, 22.16 mmol) is added. The mixture is stirred at r.t. for 30 min. The reaction is quenched with saturated NaHCO₃. The organic layer is washed with brine, dried over Na₂SO₄, concentrated to give the crude product. The crude is by Flash-Prep-HPLC. The product is dissolved in anhydrous toluene and concentrated for three times. Then the product is dissolved anhydrous acetonitrile and concentrated for three times. This resulted in 13 g to give 5-4 as a white solid. MS m/z [M−H]⁻ (ESI): 875.4. ¹H-NMR (400 MHz, CDCl₃): δ ppm 8.64-8.20 (m, 2H), 7.90-7.88 (m, 2H), 7.62-7.58 (m, 1H), 7.53-7.39 (m, 8H), 7.25-7.15 (m, 6H), 6.78-6.74 (m, 2H), 5.69 (d, J=1.72 Hz, 1H), 4.37-4.21 (m, 2H), 4.10-4.03 (m, 1H), 3.90-3.79 (m, 2H), 3.75 (d, J=1.64 Hz, 3H), 3.68-3.52 (m, 3H), 3.46-3.42 (m, 2H), 3.26 (d, J=1.2 Hz, 3H), 3.17-2.97 (m, 2H), 2.94-2.87 (m, 1H), 2.67-2.48 (m, 2H), 1.79-1.51 (m, 1H), 1.26-1.18 (m, 12H). ³¹PNMR (162 MHz, CDCl₃): 148.93, 148.03

### Example 6

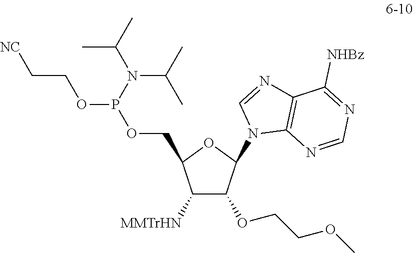

The synthesis of the 2′-O-methoxyethyl adenosine analogue 6-10 is achieved as shown below in scheme 6. The intermediate 6-2 under basic condition (NH₃/MeOH) resulted in diol 6-3, which then upon protection of 5′-hydroxy group using TBDPSCl to give 6-4 Intermediate 6-4. Then, 2′-O alkylation of 6-4 using 2-bromoethyl methyl ether/NaH/DMF to give 2′-O-methoxyethyl derivative 6-5 without the protection of C-6-exocyclic amine of 6-4. In an inventive way selective alkylation of 2′—OH group of intermediate 6-4 is achieved.

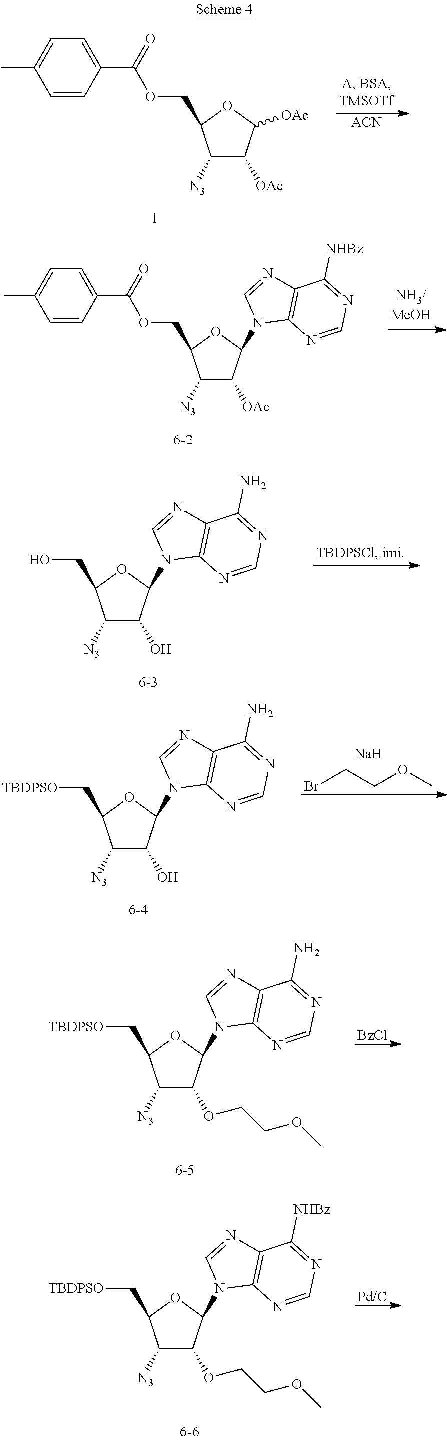

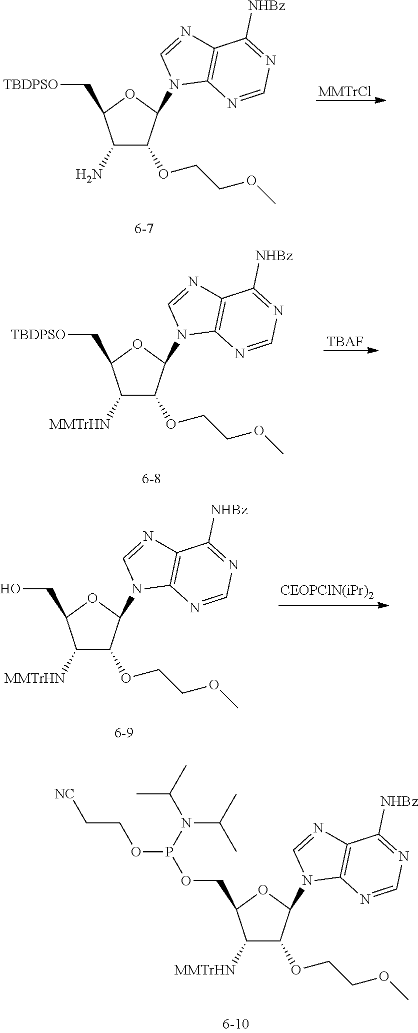

3′-Azido group of intermediate 6-5 is reduced to the amine 6-7, which is then immediately protected, such as reaction with 4-monomethoxytritylchloride, to give the precursor 6-8 after de-protection of 5′-OTBDPS group using TBAF/THF. The phosphitylation of 6-9 using known protocols is performed to give the desired 2′-O-methoxyethoxy adenine-NH-benzoyl phosphoramidite monomer 6-10.

Preparation of (6-2): To a solution of compound 1 (79.50 g, 210.68 mmol) in dry ACN (1.20 L) is added N-(5H-Purin-6-yl)benzamide (100.80 g, 421.36 mmol) and BSA (180.07 g, 884.86 mmol). The resulting suspension is stirred at 50° C. until clear. Then the mixture is cooled at −20° C. and TMSOTf (93.54 g, 421.36 mmol) is added by syringe. Then the mixture is stirred at 70° C. for 72 h under N₂, and quenched with sat NaHCO₃ and extracted with DCM. The organic layer is dried over Na₂SO₄, then solvent is evaporated, and the residue is purified on silica gel to afford compound 6-2 (107.50 g, 192.26 mmol, 91.26% yield) as a yellow solid. ¹H-NMR (400 MHz, DMSO): δ=11.28 (s, 1H), 8.64 (d, J=6.4 Hz, 2H), 8.05 (d, J=8.0 Hz, 2H), 7.84 (d, J 8.0 Hz, 2H), 7.66 (t, J=7.6 Hz, 1H), 7.56 (t, J=8.0 Hz, 2H), 7.33 (d, J=8.0 Hz, 2H), 6.37 (d, J 3.6 Hz, 1H), 6.17 (dd, J=6.0 Hz, 1H), 5.09 (t, J=6.8 Hz, 1H), 4.69-4.56 (m, 2H), 4.40-4.38 (m, 1H), 2.39 (s, 3H), 2.17 (s, 3H). ESI-LCMS: m/z 557.2 [M+H]⁺.

Preparation of (6-3): To a solution of compound 6-2 (107.50 g, 192.26 mmol) dissolved in 33 wt. % methylamine in ethanol (600.00 mL), then the mixture are stirred at 20° C. for 16 h, then solvent is evaporated, washed with 50% EtOAc in petroleum ether (1.5 L), filtered to afford compound 6-3 (52.50 g, 179.64 mmol, 93.44% yield) as a slightly yellow solid. ESI-LCMS: m/z 293.1 [M+H]⁺.

Preparation of (6-4): A solution of compound 6-3 (52.50 g, 179.64 mmol), imidazole (18.32 g, 269.46 mmol) and TBDPS-Cl (54.34 g, 197.60 mmol) in pyridine (500.00 mL) is stirred at 20° C. for 2 h, LC-MS showed 6-3 is consumed. Then quenched with MeOH (30 mL), concentrated to give the crude product which is purified on silica gel with to afford compound 6-4 (72.60 g, 136.81 mmol, 76.16% yield) as a white solid. ¹H-NMR (400 MHz, DMSO): δ=8.29 (s, 1H), 8.10 (s, 1H), 7.63-7.59 (m, 4H), 7.48-7.33 (m, 8H), 6.36 (d, J=5.6 Hz, 1H), 5.97 (d, J=4.4 Hz, 1H), 5.10-5.06 (m, 1H), 4.47 (t, J=5.6 Hz, 1H), 4.14-4.11 (m, 1H), 3.94 (dd, J=11.2 Hz, 1H), 3.83 (dd, J=11.6 Hz, 1H), 0.99 (s, 9H). ESI-LCMS: m/z 531.3 [M+H]⁺.

Preparation of (6-5): A solution of 6-4 (35.00 g, 65.96 mmol) and 1-Bromo-2-methoxyethane (18.33 g, 131.91 mmol) in dry DMF (400.00 mL), is added NaI (19.77 g, 131.91 mmol) and Ag₂O (15.29 g, 65.96 mmol), the mixture is stirred at room temperature for 5 h. Then the reaction is poured into ice water, extracted with EA, washed with brine and dried over anhydrous Na₂SO₄. The solvent is evaporated, and the residue is purified on silica gel to give 6-5 (23.70 g, 40.26 mmol, 61.04% yield) as a white solid and by-product of TBDPS lost 5.20 g, 9.81 mmol, 14.87% yield) as a white solid. ¹H-NMR (400 MHz, DMSO): δ=8.31 (s, 1H), 8.11 (s, 1H), 7.63-7.60 (m, 4H), 7.47-7.44 (m, 2H), 7.40-7.36 (m, 6H), 6.10 (d, J=4.4 Hz, 1H), 5.02 (t, J 4.8 Hz, 1H), 4.69 (t, J=5.6 Hz, 1H), 4.18-4.14 (m, 1H), 3.95 (dd, J=11.6 Hz, 1H), 3.84 (dd, J=11.6 Hz, 1H), 3.78-3.75 (m, 2H), 3.45 (t, J=4.8 Hz, 1H), 3.16 (s, 3H), 0.99 (s, 9H). ESI-LCMS: m/z 589.5 [M+H]⁺.

Preparation of (6-6): To a solution of 6-5 (31.23 g, 53.04 mmol) in pyridine (300.00 mL) at 0° C., is added BzCl (11.22 g, 79.56 mmol) dropwise. The mixture is stirred at r.t. for 2 h. Then the solution is cooled to 0° C., and ammonium hydroxide (20 mL, 30%) is added and the mixture is allowed to warm to r.t., then the solvent is evaporated, 300 mL H₂O and 600 mL EA are added into separate the solution, the aqueous is extracted by EA, combined the organic and washed with brine, dried over anhydrous Na₂SO₄, the solvent is removed and the residue is purified on silica gel to give 6-6 (28.70 g, 41.42 mmol, 78.09% yield) as a white solid. ESI-LCMS: m/z 693.4 [M+H]⁺.

Preparation of (6-7): A solution of 6-6 (28.70 g, 41.42 mmol) in EA (150.00 mL) is added Pd/C (3.00 g) and MeOH (150.00 mL) under H₂. The mixture is stirred at r.t. for 5 h. Then the reaction is filtered and the filtrate concentrated to give 6-7 (25.49 g, 38.22 mmol, 92.27% yield) as a gray solid. ESI-LCMS: m/z 667.3 [M+H]⁺.

Preparation of (6-8): To a solution of 6-7 (25.49 g, 38.22 mmol) and AgNO₃ (12.98 g, 76.44 mmol) in DCM (300.00 mL) is added collidine (13.89 g, 114.66 mmol) and MMTrCl (19.43 g, 57.33 mmol), the mixture is stirred at r.t. for 2 h. Then the reaction is poured into ice water, the organic layer extracted with DCM, washed with brine and dried over anhydrous Na₂SO₄, the solvent is removed and the residue is purified on silica gel to give 6-8 (32.79 g, 34.92 mmol, 91.36% yield) as a gray solid.

Preparation of (6-9): A solution of 6-8 (32.79 g, 34.92 mmol) in THF (300.00 mL) is added TBAF (1M, 35.00 mL), the mixture is stirred at room temperature for 15 h. Then the solvent is removed and the residue is purified on silica gel with EA to give 6-9 (22.22 g, 31.71 mmol, 90.82% yield) as a white solid. ¹H-NMR (400 MHz, CDCl₃): δ=8.68 (s, 1H), 8.32 (s, 1H), 8.04 (d, J=7.2 Hz, 2H), 7.61-7.57 (m, 1H), 7.53-7.48 (m, 6H), 7.40 (d, J=8.8 Hz, 2H), 7.21-7.12 (m, 6H), 6.73 (d, J=8.8 Hz, 2H), 6.09 (d, J=2.4 Hz, 2H), 4.08-4.02 (m, 2H), 3.93-3.87 (m, 1H), 3.72 (s, 3H), 3.58-3.53 (m, 1H), 3.43-3.39 (m, 3H), 3.24-3.19 (m, 4H), 2.19 (br, 1H).

Preparation of (6-10): To a solution of 6-9 (14.00 g, 19.98 mmol), DMAP (488.19 mg, 4.00 mmol) and DIPEA (6.46 g, 49.95 mmol, 8.73 mL) in dry DCM (100.00 mL) is added CEPCl (5.68 g, 23.98 mmol) dropwise under Ar. The mixture is stirred at room temperature for 1 h. Then the reaction is wished with 10% NaHCO₃(aq) and brine, dried over Na₂SO₄, the solvent is removed and the residue is purified by c.c. with the PE/EA mixture, then concentrated to give the crude product. The crude product (10 g, dissolved in 10 mL of ACN) is purified by Flash-Prep-HPLC to obtain 6-10 (12.60 g, 13.98 mmol, 69.99% yield) as a white solid. Then the product is dissolved in dry toluene (15 mL) and concentrated three times, and with dry ACN three times. ¹H-NMR (400 MHz, CDCl₃): δ=9.12 (d, J=46.8 Hz, 1H), S=8.71 (d, J=11.6 Hz, 1H), 8.50 (s, 0.6H), 8.22 (s, 0.4H), 8.04 (t, J=7.2 Hz, 2H), 7.63-7.59 (m, 1H), 7.55-7.46 (m, 6H), 7.40-7.37 (m, 2H), 7.19-7.06 (m, 6H), 6.69 (dd, J=8.8 Hz, 2H), 6.03 (d, J=3.2 Hz, 1H), 4.36-4.24 (m, 2H), 3.92-3.78 (m, 2H), 3.71 (d, J=11.6 Hz, 3H), 3.67-3.33 (m, 7H), 3.29 (d, J=11.2 Hz, 3H), 3.17-3.10 (m, 1H), 2.88 (dd, J=27.2 Hz, 1H), 2.65-2.50 (m, 2H), 2.38 (d, J=4.4 Hz, 0.4H), 1.80 (d, J=4.0 Hz, 0.6H), 1.23-1.15 (m, 12H). ³¹PNMR (400 MHz, CDCl₃): 148.86, 148.22. ESI-LCMS: m/z 901.3 [M+H]⁺.

### Quantitation of Crude Oligomer or Raw Analysis

Samples are dissolved in deionized water (1.0 mL) and quantitated as follows: Blanking is first performed with water alone (1.0 mL) 20 ul of sample and 980 μL of water are mixed well in a microfuge tube, transferred to cuvette and absorbance reading obtained at 260 nm. The crude material is dried down and stored at −20° C.

### Crude HPLC/LC-MS Analysis

The 0.1 OD of the crude samples are submitted for crude MS analysis. After Confirming the crude LC-MS data then purification step is performed.

### HPLC Purification

The Phosphoramidate (NP) and Thiophosphoramidate (NPS) modified oligonucleotides with and without GalNAc conjugates are purified by anion-exchange HPLC. The buffers are 20 mM sodium phosphate in 10% CH₃CN, pH 8.5 (buffer A) and 20 mM sodium phosphate in 10% CH₃CN, 1.8 M NaBr, pH 8.5 (buffer B). Fractions containing full-length oligonucleotides are pooled, desalted, and lyophilized.

### Desalting of Purified Oligomer

The purified dry oligomer is then desalted using Sephadex G-25 M (Amersham Biosciences). The cartridge is conditioned with 10 mL of deionized water thrice. Finally the purified oligomer dissolved thoroughly in 2.5 mL RNAse free water is applied to the cartridge with very slow drop wise elution. The salt free oligomer is eluted with 3.5 ml deionized water directly into a screw cap vial.

### IEX HPLC and Electrospray LC/MS Analysis

Approximately 0.10 OD of oligomer is dissolved in water and then pipetted in special vials for IEX-HPLC and LC/MS analysis. Analytical HPLC and ES LC-MS established the integrity of the oligonucleotides. The purity and molecular weight are determined by HPLC analysis (60° C., IEX-Thermo DNAPac PA-100, A-25 mM sodium phosphate 10% acetonitrile pH 11, B— 1.8 M NaBr 25 mM sodium phosphate 10% acetonitrile pH 11; RPIP-Waters XBridge OST C18, A-100 mM HFIP 7 mM TEA B— 7:3 methanol/acetonitrile) and ESI-MS analysis using Promass Deconvolution for Xcalibur (Novatia, Newtown, Pa.). All oligonucleotides in the following tables are synthesized, and reference to molecular weights in the tables are actual measured weights that may have an error of MW, amu+/−2.

### In Vitro Testing of Oligonucleotides

An HBV cell line is used to assess the in vitro potency of NAPs: HepG2.2.15 (2215). HBsAg reduction in tissue culture supernatant (sup) as well as cytotoxicity is measured using HepG2.2.15 cell.

HepG2.2.15 cell line is a stable cell line with four integrated HBV genomes. The cells are grown at 37° C. in an atmosphere of 5% C02 in Dulbecco's modified Eagle's medium supplemented with 10% FCS, 100 IU/ml penicillin, 100 μg/ml streptomycin, and 2% glutamine. The day before the dosing, 2.5×10⁴ cells/well are plated in collagen coated 96 well plates and incubated overnight.

On the day of dosing, serially diluted oligomers are transfected into the cells with Lipofectamine RNAiMax (Thermo Fisher, Waltham, Mass.) following manufacturer's protocol. Duplicates are made for each drug concentration and each NAP is set up for both EC50 measurement and CC50 measurement. Three days after transfection, the supernatant (sup) is collected and used in HBsAg ELISA (AutoBio, China) for EC50 calculation. For CC50 measurement, CellTiter-Glo® (Promega, Madison, Wis.) is used in the assay following manufacturer's instruction.

Resulting EC50 and CC50 are shown in the following tables.

| | HepG 2.2.15 | |
|---|---|---|
| Sequence | EC50 (μM) | CC50 (μM) |
| (moeAps(5m)mCps)20 | 0.37 | >15 |
| (mAps(5m)mCps)20 | 6.55/4.39 | >15 |

As can be seen above, replacement of 2′OMeA with 2′MOEA resulted in a significant improvement in EC50.

| | HepG 2.2.15 | |
|---|---|---|
| Sequence | EC50 (μM) | CC50 (μM) |
| (5m)mCnpsmApsmmCpsmAps)10 | 0.23 | >15 |
| (mApsmmCpsmApsmmCnps)10 | 0.27 | >15 |
| (mAnps(5m)mCpsmAps(5m)mCps)10 | 0.79 | >15 |
| (AnpsCnps)15 | 0.03 | >15 |
| (AnpsCnps)12 | 0.77 | >15 |
| (AnpsCnps)9 | 0.61 | >15 |
| (AnpsCnps)6 | 0.73 | >15 |
| (mApsmmCps)20 | 6.55/4.39 | >15 |

As can be seen above, incorporation of nps intersubunit linkages resulted in a significant improvement in EC50. Furthermore, a significant improvement in EC50 was maintained, even in shorter NAPs.

### EQUIVALENTS

The present disclosure is not to be limited in terms of the particular embodiments described in this application, which are intended as single illustrations of individual aspects of the present disclosure. Many modifications and variations of this present disclosure can be made without departing from its spirit and scope, as will be apparent to those skilled in the art. Functionally equivalent methods and apparatuses within the scope of the present disclosure, in addition to those enumerated herein, will be apparent to those skilled in the art from the foregoing descriptions. Such modifications and variations are intended to fall within the scope of the present disclosure. It is to be understood that this present disclosure is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A nucleic acid polymer comprising 8 to 50 nucleoside subunits linked by intersubunit linkages, wherein the nucleic acid polymer comprises (A) one or more 3′-5′ thiophosphoramidate intersubunit linkage and the remaining intersubunit linkages are 3′-5′ thiophosphate intersubunit linkages and/or (B) at least 40% of the nucleoside subunits contain a 2′-MOE substituent.

2. The nucleic acid polymer of claim 1, wherein the nucleic acid polymer comprises five or more 3′-5′ thiophosphoramidate intersubunit linkages and the remaining intersubunit linkages are 3′-5′ thiophosphate intersubunit linkages.

3. The nucleic acid polymer of claim 1, wherein the nucleic acid polymer, wherein half the intersubunit linkages are 3′-5′ thiophosphoramidate intersubunit linkages and the remaining intersubunit linkages are 3′-5′ thiophosphate intersubunit linkages.

4. The nucleic acid polymer of claim 1, wherein the nucleoside subunits each independently contain a nucleobase selected from adenine, guanine, cytosine, 5-methylcytosine, and uracil.

5. The nucleic acid polymer of claim 4, wherein the nucleobase is selected from adenine, cytosine, and 5-methylcytosine.

6. The nucleic acid polymer of claim 5, wherein the nucleobase is selected from adenine, cytosine, and 5-methylcytosine.

7. The nucleic acid polymer of claim 1, wherein the nucleoside subunits are substituted at the 2′ position with OMe or MOE.

8. The nucleic acid polymer of claim 1, wherein the nucleic acid polymer is represented by the following formula (I):
(N¹-L¹-N²-L²-N³-L³-N⁴-L⁴)ₓ (I)
wherein
N¹ and N³ represent a nucleoside with a nucleobase selected from adenine, guanine, cytosine, 5-methylcytosine, and uracil;
N² and N⁴ represent a nucleoside with a nucleobase selected from adenine, guanine, cytosine, 5-methylcytosine, and uracil;
L¹, L², L³ and L⁴ each independently are a ps or nps linkage, and at least one is a nps linkage; and
x is an integer from 2 to 16.

9. The nucleic acid polymer of claim 8, wherein N¹, N², N³ and N⁴ are each independently substituted at the 2′ position with OMe or MOE.

10. The nucleic acid polymer of claim 8, wherein the nucleobase of N¹ and N³ is adenine and the nucleobase of N² and N⁴ is cytosine or 5-methylcytosine.

11. The nucleic acid polymer of claim 8, wherein one of L¹ and L² is nps.

12. The nucleic acid polymer of claim 8, wherein two of L¹, L², L³ and L⁴ is nps.

13. The nucleic acid polymer of claim 8, wherein x is an integer from 2 to 10.

14. The nucleic acid polymer of claim 1, wherein the nucleic acid polymer is represented by the following formula (II):
(N⁵-L⁵-N⁶-L⁶)_{y} (II)
wherein
N⁵ and N⁶ represent a nucleoside with a nucleobase selected from adenine, guanine, cytosine, 5-methylcytosine, and uracil;
L⁵ and L⁶ each independently are a ps or nps linkage, and at least one is a nps linkage; and
x is an integer from 4 to 22.

15. The nucleic acid polymer of claim 14, wherein N⁵ and N⁶ are each independently substituted at the 2′ position with OMe or MOE.

16. The nucleic acid polymer of claim 14, wherein the nucleobase of N⁵ is adenine and the nucleobase of N⁶ is cytosine or 5-methylcytosine.

17. The nucleic acid polymer of claim 14, wherein L⁵ and L⁶ are each nps.

18-19. (canceled)

20. The nucleic acid polymer of claim 1, wherein the nucleic acid polymer is selected from:
(mXnpsmXpsmXpsmXps)10;
(mXpsmXnpsmXpsmXps)10;
(mXpsmXpsmXnpsmXps)10;
(mXpsmXpsmXpsmXnps)10;
(XnpsXnps)5-15; and
(moeXpsmXps)20,
wherein X is independently in each instance a nucleoside having natural or modified nucleobase.

21. A pharmaceutical composition comprising a nucleic acid polymer of claim 1 and a pharmaceutically acceptable excipient.

22. A method for treating a subject having a viral infection comprising administering to the subject in need thereof a therapeutically effective amount of the nucleic acid polymer of claim 1.

23-33. (canceled)
